(19) United States
(12) Patent Application Publication
(10) Pub. No.: US 2003/0119859 A1
(43) Pub. Date: Jun. 26, 2003

(54) Medical combinations comprising tiotropium and rofleponide

(76) Inventor: Brian Charles Gavin, Rathfarnfam (IE)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/257,703

(22) PCT Filed: Apr. 11, 2001
(86) PCT No.: PCT/GB01/01627

(30) Foreign Application Priority Data
Apr. 18, 2000 (GB) 0009592.7

(51) Int. Cl. A61L 9/04; A61K 31/4745

(52) U.S. Cl. 514/291; 424/45

(57) ABSTRACT

The present invention is concerned with pharmaceutical formulations comprising a combination of tiotropium and rofleponide and the use of such formulations in medicine, particularly in the prophylaxis and treatment of respiratory diseases.

The present invention is concerned with combinations of tiotropium and rofleponide, particularly compositions containing a combination of tiotropium and rofleponide and the use of such compositions in medicine, particularly in the prophylaxis and treatment of respiratory diseases.

Tiotropium i.e. (1α, 2β, 4β, 5α, 7β)-7-[(hydroxydi-2-thienylacetyl)oxy]-9,9-dimethyl-3-oxa-9-azoniatricyclo[3.3.2.0]nonane and particularly its bromide salt is a well-known anti-cholinergic agent, described in EP418,716 for the treatment of bronchial asthma and related disorders.

WO 92/13872 describes rofleponide i.e. 16α, 17α-butylidenedioxy-6α, 9α-difluoro-11β, 21-dihydroxypregn-4-ene-3,20-dione, salts and esters thereof and pharmaceutical formulations thereof. Rofleponide is an antiinflammatory corticosteroid, which is proposed for use in the treatment of bronchial asthma and related disorders.

Although tiotropium bromide and rofleponide may be effective therapies, there exists a clinical need for asthma therapies having potent and selective action and having an advantageous profile of action.

Therefore, according to the present invention there is provided a combination of tiotropium or a pharmaceutically acceptable salt, solvate, or physiologically functional derivative thereof and rofleponide or a pharmaceutically acceptable salt, solvate, or physiologically functional derivative thereof.

It will be appreciated that the compounds of the combination may be administered simultaneously, either in the same or different pharmaceutical formulations or sequentially. If there is sequential administration, the delay in administering the second compound should not be such as to lose the beneficial therapeutic effect of the combination.

According to a further aspect of the present invention, there is provided a pharmaceutical formulation comprising tiotropium or a pharmaceutically acceptable salt, solvate, or physiologically functional derivative thereof and rofleponide or a pharmaceutically acceptable salt, solvate, or physiologically functional derivative thereof, and a pharmaceutically acceptable carrier or excipient, and optionally one or more other therapeutic ingredients. According to a preferred aspect of the present invention, there is provided a pharmaceutical formulation comprising tiotropium bromide and rofleponide, and a pharmaceutically acceptable carrier or excipient, and optionally one or more other therapeutic ingredients. In the most preferred aspect, the above pharmaceutical formulations are suitable for administration by inhalation.

It is to be understood that the present invention covers all combinations of particular and preferred aspects of the invention described herein.

As would be appreciated by the skilled person, rofleponide contains several asymmetric centres. The present invention includes each isomer of rofleponide, particularly the (22R) and (22S) isomers either in substantially pure form or admixed in any proportions. The isomers of rofleponide have been described previously in WO 92/13872.

By the term “physiologically functional derivative” is meant a chemical derivative of tiotropium or rofleponide having the same physiological function as the free compound, for example, by being convertible in the body thereto. According to the present invention, examples of physiologically functional derivatives include esters.

Suitable salts according to the invention include those formed with both organic and inorganic acids. Pharmaceutically acceptable acid addition salts include but are not limited to those formed from hydrochloric, hydrobromic, sulphuric, citric, tartaric, phosphoric, lactic, pyruvic, acetic, trifluoroacetic, succinic, oxalic, fumaric, maleic, oxaloacetic, methanesulphonic, ethanesulphonic, p-toluenesulphonic, benzenesulphonic, isethionic, and naphthalenecarboxylic, such as 1-hydroxy-2-naphthalenecarboxylic acids.

Pharmaceutically acceptable esters of tiotropium or rofleponide may have a hydroxyl group converted to a C₁₋₆-alkyl, aryl, aryl C₁₋₆alkyl, or amino acid ester.

As mentioned above, both tiotropium and rofleponide and their pharmaceutically acceptable salts, solvates, and physiologically functional derivatives have been described for use in the treatment of respiratory diseases. Therefore, formulations of tiotropium and rofleponide and their pharmaceutically acceptable salts, solvates, and physiologically functional derivatives have use in the prophylaxis and treatment of clinical conditions for which anticholinergic agent and/or an antiinflammatory corticosteroid is indicated. Such conditions include diseases associated with reversible airways obstruction such as asthma, chronic obstructive pulmonary diseases (COPD) (e.g. chronic and wheezy bronchitis, emphysema), respiratory tract infection and upper respiratory tract disease.

Accordingly, the present invention provides a method for the prophylaxis or treatment of a clinical condition in a mammal, such as a human, for which an anticholinergic agent and/or antiinflammatory corticosteroid is indicated, which comprises administration of a therapeutically effective amount of a combination of tiotropium or a pharmaceutically acceptable salt, solvate, or physiologically functional derivative thereof and rofleponide or a pharmaceutically acceptable salt, solvate, or physiologically functional derivative thereof. The present invention further provides a method for the prophylaxis or treatment of a clinical condition in a mammal, such as a human, for which an anticholinergic agent and/or antiinflammatory corticosteroid is indicated, which comprises administration of a therapeutically effective amount of a pharmaceutical formulation comprising tiotropium or a pharmaceutically acceptable salt, solvate, or physiologically functional derivative thereof and rofleponide or a pharmaceutically acceptable salt, solvate, or physiologically functional derivative thereof, and a pharmaceutically acceptable carrier or excipient. In a preferred aspect, there is provided such a method which comprises administration of a therapeutically effective amount of a pharmaceutical formulation comprising tiotropium bromide and rofleponide, and a pharmaceutically acceptable carrier or excipient. In particular, the present invention provides such methods for the prophylaxis or treatment of a disease associated with reversible airways obstruction such as asthma, chronic obstructive pulmonary disease (COPD), respiratory tract infection or upper respiratory tract disease.

In the alternative, there is provided a combination of tiotropium or a pharmaceutically acceptable salt, solvate, or physiologically functional derivative thereof and rofleponide or a pharmaceutically acceptable salt, solvate, or physiologically functional derivative thereof, for use in therapy, particularly for use in the prophylaxis or treatment of a clinical condition for which an anticholinergic agent and/or antiinflammatory corticosteroid is indicated. In particular, there is provided a pharmaceutical formulation comprising tiotropium or a pharmaceutically acceptable salt, solvate, or physiologically functional derivative thereof (suitably, tiotropium bromide) and rofleponide or a pharmaceutically acceptable salt, solvate, or physiologically functional derivative thereof, and a pharmaceutically acceptable carrier or excipient for use in therapy, particularly for use in the prophylaxis or treatment of a clinical condition for which an anticholinergic agent and/or antiinflammatory corticosteroid is indicated. In a preferred aspect, the invention is concerned with the prophylaxis or treatment of a disease associated with reversible airways obstruction such as asthma, chronic obstructive pulmonary disease (COPD), respiratory tract infection or upper respiratory tract disease.

The amount of tiotropiumr and rofleponide, or a pharmaceutically acceptable salt, solvate or physiologically functional derivative thereof which is required to achieve a therapeutic effect will, of course, vary with the particular compound, the route of administration, the subject under treatment, and the particular disorder or disease being treated. As a monotherapy, tiotropium bromide is generally administered to adult humans by aerosol inhalation at a dose of 10 mcg to 200 mcg twice daily. As a monotherapy, rofleponide is described in WO 92/13872 as being administered to adult humans by aerosol inhalation at a dose of from 10 mcg to 1000 mcg, preferably 20 mcg to 250 mcg.

While it is possible for the active ingredients of the combination to be administered as the raw chemical, it is preferable to present them as a pharmaceutical formulation. When the individual compounds of the combination are administered separately, they are generally each presented as a pharmaceutical formulation as described previously in the art.

Pharmaceutical formulations are often prescribed to the patient in “patient packs” containing the whole course of treatment in a single package. Patient packs have an advantage over traditional prescriptions, where a pharmacist divides a patient'supply of a pharmaceutical from a bulk supply, in that the patient always has access to the package insert contained in the patient pack, normally missing in traditional prescriptions. The inclusion of a package insert has been shown to improve patient compliance with the physician'instructions and, therefore, lead generally to more successful treatment. It will be understood that the administration of the combination of the invention by means of a single patient pack, or patient packs of each component compound, and containing a package insert instructing the patient to the correct use of the invention is a desirable additional feature of the invention.

Hereinafter, the term “active ingredients” means tiotropium or a pharmaceutically acceptable salt, solvate, or physiologically functional derivative thereof, preferably tiotropium bromide, and rofleponide, or a pharmaceutically acceptable salt, solvate, or physiologically functional derivative thereof.

Suitably, the pharmaceutical formulations which are suitable for inhalation according to the invention comprise the active ingredients in amounts such that each actuation provides therapeutically effective dose, for example, a dose of tiotropium of 10 mcg to 200 mcg, preferably 20 mcg to 100 mcg and a dose of rofleponide of 10 mcg to 1.6mg, preferably 20 mcg to 250 mcg.

The pharmaceutical formulations according to the invention may further include other therapeutic agents for example anti-inflammatory agents such as other corticosteroids (e.g. fluticasone propionate, beclomethasone dipropionate, mometasone furoate, triamcinolone acetonide or budesonide) or NSAIDs (e.g. sodium cromoglycate, nedocromil sodium; PDE-4 inhibitors, leukotriene antagonists, iNOS inhibitors, tryptase and elastase inhibitors, beta-2 integrin antagonists and adenosine 2a agonists), or, β₂-adrenoreceptor agonists (such as salbutamol, formoterol, salmeterol, fenoterol or terbutaline and salts thereof), or other anticholinergic agents (such as ipratropium).

The formulations include those suitable for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the active ingredients into association with the carrier which constitutes one or more accessory ingredients. In general the formulations are prepared by uniformly and intimately bringing into association the active ingredients with liquid carriers or finely divided solid carriers or both and then, if necessary, shaping the product into the desired formulation.

Formulations for inhalation include powder compositions which will preferably contain lactose, and spray compositions which may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluoroethane, carbon dioxide or other suitable gas. Suitable aerosol formulations include those described in EP 0372777 and WO93/11743. For suspension aerosols, the active ingredients should be micronised so as to permit inhalation of substantially all of the active ingredients into the lungs upon administration of the aerosol formulation, thus the active ingredients will have a particle size of less than 100 microns, desirably less than 20 microns, and preferably in the range 1 to 10 microns, for example, 1 to 5 microns.

Intranasal sprays may be formulated with aqueous or non-aqueous vehicles with the addition of agents such as thickening agents, buffer salts or acid or alkali to adjust the pH, isotonicity adjusting agents or anti-oxidants.

Capsules and cartridges or for example gelatin, or blisters of for example laminated aluminium foil, for use in an inhaler or insuflator may be formulated containing a powder mix of the active ingredients and a suitable powder base such as lactose or starch. In this aspect, the active ingredients are suitably micronised so as to permit inhalation of substantially all of the active ingredients into the lungs upon administration of the dry powder formulation, thus the active ingredients will have a particle size of less than 100 microns, desirably less than 20 microns, and preferably in the range 1 to 10 microns.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Preferred unit dosage formulations are those containing a pharmaceutically effective dose, as hereinbefore recited, or an appropriate fraction thereof, of the active ingredient. Thus, in the case of formulations designed for delivery by metered dose pressurised aerosols, one actuation of the aerosol may deliver half of the therapeutically effective amount such that two actuations are necessary to deliver the therapeutically effective dose.

It should be understood that in addition to the ingredients particularly mentioned above, the formulations of this invention may include other agents conventional in the art having regard to the type of formulation in question. Furthermore, the claimed formulations include bioequivalents as defined by the US Food. and Drugs Agency.

For a better understanding of the invention, the following Examples are given by way of illustration.

### EXAMPLES

A: Metered Dose Inhalers

### Example 1

| | Per actuation |
|---|---|
| tiotropium bromide | 100 microgram |
| rofleponide | 200 microgram |
| 1,1,1,2-Tetrafluoroethane | to 75.0 mg |

The micronised active ingredients are weighed into an aluminium can, 1,1,1,2-tetrafluoroethane is then added from a vacuum flask and a metering valve is crimped into place.

Similar methods may be used for the formulation of Examples 2 to 4:

### Example 2

| | Per actuation |
|---|---|
| tiotropium bromide | 200 microgram |
| rofleponide | 100 microgram |
| 1,1,1,2-Tetrafluoroethane | to 75.0 mg |

### Example 3

| | Per actuation |
|---|---|
| tiotropium bromide | 18 microgram |
| rofleponide | 100 microgram |
| 1,1,1,2-Tetrafluoroethane | to 75.0 mg |

### Example 4

| | Per actuation |
|---|---|
| tiotropium bromide | 18 microgram |
| rofleponide | 200 microgram |
| 1,1,1,2-Tetrafluoroethane | to 75.0 mg |

B: Dry Powder Inhalers

### Example 5

| | Per cartridge or blister |
|---|---|
| tiotropium bromide | 100 microgram |
| rofleponide | 200 microgram |
| Lactose Ph. Eur. | to 12.5 mg |
| | or to 25.0 mg |

The active ingredients are micronised and bulk blended with the lactose in the proportions given above. The blend is filled into hard gelatin capsules or cartridges or in specifically constructed double foil blister packs to be administered by an inhaler such as a Rotahaler, Diskhaler, or Diskus inhaler (each of these being a Trademark of Glaxo Group Limited).

Similar methods may be used for the formulations of Examples 6 to 8:

### Example 6

| | Per cartridge or blister |
|---|---|
| tiotropium bromide | 200 microgram |
| rofleponide | 100 microgram |
| Lactose Ph. Eur. | to 12.5 mg |
| | or to 25.0 mg |

### Example 7

| | Per cartridge or blister |
|---|---|
| tiotropium bromide | 18 microgram |
| rofleponide | 100 microgram |
| Lactose Ph. Eur. | to 12.5 mg |
| | or to 25.0 mg |

### Example 8

| | Per cartridge or blister |
|---|---|
| tiotropium bromide | 18 microgram |
| rofleponide | 200 microgram |
| Lactose Ph. Eur. | to 12.5 mg |
| | or to 25.0 mg |

1. A pharmaceutical formulation comprising tiotropium or a pharmaceutically acceptable salt, solvate, or physiologically functional derivative thereof and rofleponide or a pharmaceutically acceptable salt, solvate, or physiologically functional derivative thereof, and a pharmaceutically acceptable carrier or excipient, and optionally one or more other therapeutic ingredients.

2. A pharmaceutical formulation comprising tiotropium bromide and rofleponide, and a pharmaceutically acceptable carrier or excipient, and optionally one or more other therapeutic ingredients.

3. A pharmaceutical formulation comprising tiotropium bromide and rofleponide palmitate, and a pharmaceutically acceptable carrier or excipient, and optionally one or more other therapeutic ingredients.

4. A pharmaceutical formulation according to any of claims 1 to 3 which is suitable for administration by inhalation.

5. A pharmaceutical formulation according to any of claims 1 to 4 wherein the pharmaceutically acceptable carrier or excipient is lactose.

6. A pharmaceutical formulation according to any of claims 1 to 4 wherein the pharmaceutically acceptable carrier or excipient comprises 1,1,1,2-tetrafluoroethane and/or 1,1,1,2,3,3,3-heptafluoropropane.

7. A method for the prophylaxis or treatment of a clinical condition in a mammal, such as a human, for which an anticholinergic agent and/or antiinflammatory corticosteroid is indicated, which comprises administration of a therapeutically effective amount of a pharmaceutical formulation according to any one of claims 1 to 37

8. A method according to claim 7 wherein the clinical condition is a disease associated with reversible airways obstruction such as asthma, chronic obstructive pulmonary disease (COPD), respiratory tract infection or upper respiratory tract disease.
